(19) United States
(12) Patent Application Publication
(10) Pub. No.: US 2025/0241654 A1
(43) Pub. Date: Jul. 31, 2025

(54) TOURNIQUET CONTROL USING VISUALIZATION SYSTEM

(71) Applicant: Relign Corporation, Campbell, CA (US)

(72) Inventor: Aaron Germain, San Jose, CA (US)

(21) Appl. No.: 19/036,437

(22) Filed: Jan. 24, 2025

Related U.S. Application Data
(60) Provisional application No. 63/626,221, filed on Jan. 29, 2024.

(51) Int. Cl. A61B 17/132; A61B 17/00; G16H 50/70

(52) U.S. Cl. CPC A61B 17/132; G16H 50/70; A61B 2017/00022

(57) ABSTRACT

A surgical system optionally includes a tourniquet system, a visualization system and a controller. The tourniquet system including a cuff configured to perform occlusion of a limb of a patient during a surgical procedure. The visualization system including one or more cameras configured to provide a field of view that includes, a surgical site at the limb of the patient. The controller electronically coupled to the visualization system and the tourniquet system. The controller is configured to determine a presence or an absence of bleeding at the surgical site based upon data from the visualization system, and control the tourniquet system to increase or decrease of a pressure of the cuff in response to such determination.

### CLAIM OF PRIORITY

This application claims the benefit of U.S. Provisional Patent Application Ser. No. 63/626,221, filed on Jan. 29, 2024, the benefit of priority of which is claimed hereby, and which is incorporated by reference herein in its entirety.

### FIELD OF THE INVENTION

The present invention relates generally to surgical systems and methods that use imaging during a surgical procedure to visualize a presence of or absence of bleeding and control a tourniquet in response to such visualizing.

### BACKGROUND

Arthroscopic surgery is typically a minimally invasive procedure performed through small incisions for sports medicine. Arthroplasty can be a more extensive procedure involving replacement of all or a portion of a joint. Tourniquets are commonly used in such surgical procedures to control bleeding by compressing blood vessels and restricting blood flow to the area of surgery. This is typically achieved by applying a pressure cuff to the patient's limb above the surgical site. The pressure applied by the cuff is critical; it must be sufficient to prevent bleeding without causing tissue damage or other complications.

Traditionally, the pressure applied by a tourniquet is determined using limb occlusion pressure (LOP) calculations, which can be derived from separate sensors or manual calculations performed by medical staff. However, this approach does not provide real-time feedback on actual bleeding at the surgical site, and can potentially result in over- or under-pressurization of the tourniquet to meet actual bleeding needs.

### OVERVIEW

There is a need for a surgical system that includes a tourniquet system that is responsive to feedback regarding bleeding at the surgical site from a visualization system. The present inventor has recognized such surgical systems. These surgical systems can better prevent over-pressurization of a patient's limb. Over-pressurization as a result of an over-pressure cuff can result in postoperative complications, including pain, nerve injury, etc. Conversely, under-pressurization may lead to excessive bleeding, which can obscure the surgical field and increase the risk of complications. The present inventor recognizes the lack of dynamic, responsive control over tourniquet pressure represents a significant limitation in the current state of the art.

The present inventor also recognizes many existing surgical systems do not incorporate advanced technologies such as machine learning algorithms that can learn from past surgical outcomes to improve decision-making such as the application of suitable pressure to a cuff during surgery. The integration of such technologies into tourniquet systems such as those disclosed herein has the potential to enhance surgical precision and patient safety.

In light of these challenges, there is a need for an improved surgical systems including a tourniquet system that can dynamically adjust based upon visual feedback to the real-time conditions of the surgical site, thereby optimizing patient outcomes and reducing the risk of tourniquet-related complications. Thus, the disclosed surgical systems can allow for more effective real-time feedback for surgeons.

Various relevant arthroplasty and arthroscopic instruments including some with visualization capability that can be used with the systems and methods disclosed herein are commonly owned by the applicant. These instruments can be used discussed herein. Relevant patent publications include: U.S. Pat. Nos. 11,065,023; 11,172,953; US 2018-0303509; US 2019-0008541; US 2019-0059983; US 2019-0134279; US 2019-0021788; US 2018-0317957; US 2019-0008538; US 2019-0083121; US 2018-0263649; US 2017-0290602 and US 2019-0015151, the full disclosures of each of which are incorporated herein by reference. Relevant patent applications include U.S. Provisional Patent Application 63/588,377, entitled “ARTHROSCOPIC DEVICES AND METHODS”, filed Oct. 6, 2023; U.S. Provisional Patent Application 63/588,382, entitled “SURGICAL VISUALIZATION ASSEMBLY AND SYSTEM”, filed Oct. 6, 2023; and U.S. Provisional Patent Application 63/588,386, “ELECTROSURGICAL DEVICES AND SYSTEMS”, filed Oct. 6, 2023, the full disclosures of each of which are incorporated herein by reference.

The following, non-limiting examples, detail certain aspects of the present subject matter to solve the challenges and provide the benefits discussed herein, among others.

Example 1 is a surgical system optionally comprising: a tourniquet system including a cuff configured to perform occlusion of a limb of a patient during a surgical procedure; a visualization system including one or more cameras configured to provide a field of view that includes, a surgical site at the limb of the patient; and a controller electronically coupled to the visualization system and the tourniquet system, wherein the controller is configured to determine a presence or an absence of bleeding at the surgical site based upon data from the visualization system, and control the tourniquet system to increase or decrease of a pressure of the cuff in response to such determination.

In Example 2, the subject matter of Example 1 optionally includes, wherein the tourniquet system includes at least one sensor configured to sense an arterial blood pressure at the limb of the patient, and wherein the controller is configured to control the tourniquet system to increase or decrease the pressure of the cuff in response to data from the at least one sensor in addition to the data from the visualization system.

In Example 3, the subject matter of Examples 1-2 optionally includes, wherein the visualization system is coupled to and integrated in an arthroscopic device configured for use during the surgical procedure.

In Example 4, the subject matter of Examples 1-3 optionally includes, if the presence of bleeding is determined, the controller is configured to increase the pressure of the cuff by between 10% and 25%, inclusive, and the controller is configured to redetermine based upon second data from the visualization system gathered after an elapsed period of time, the presence or the absence of bleeding at the surgical site.

In Example 5, the subject matter of Examples 1-4 optionally includes, wherein, if the absence of bleeding is determined, the controller is configured to decrease the pressure of the cuff by between 10% and 25%, inclusive, and the controller is configured to redetermine based upon second data from the visualization system gathered after an elapsed period of time, the presence or the absence of bleeding at the surgical site.

In Example 6, the subject matter of Examples 1-5 optionally includes, wherein the controller determines the presence or absence of bleeding based as a percent of red color pixels at the surgical site and a rate of change of the red color pixels.

In Example 7, the subject matter of Examples 1-6 optionally includes, a flow inducing device configured to provide an irrigating fluid to the surgical site; and a negative pressure source configured to induce flow to remove the irrigating fluid from the surgical site; wherein the controller is configured to selectively control the flow inducing device and the negative pressure source to increase or decrease irrigating fluid at the surgical site based upon the determined presence or absence of bleeding at the surgical site.

In Example 8, the subject matter of Example 7 optionally includes, wherein the controller is configured to: collect and record the data from the visualization system during at least a portion of the surgical procedure; query a database to retrieve information about related prior surgical procedures, the information including at least one result or next action taken after the data from the visualization system was provided to the controller; determine, based upon the information, a next action including at least one of: implementing a change of the pressure of the cuff of the tourniquet system and implementing an addition or removal of the irrigating fluid from the surgical site; implement the next action; and collect further data from at least the visualization system as to results from the next action.

In Example 9, the subject matter of Examples 1-8 optionally includes, wherein the controller is configured to: collect and record the data from the visualization system during at least portion of the surgical procedure; query a database to retrieve information about related prior surgical procedures, the information including at least one result or next action taken after the data from the visualization system was provided to the controller; determine, based upon the information, a change of the pressure of the cuff of the tourniquet system; implement the change; and collect further data from at least the visualization system and redetermine the presence or absence of bleeding as a result of the change.

In Example 10, the subject matter of Examples 8-9 optionally includes, a machine learning engine and wherein the controller is configured to train the machine learning engine using the related prior surgical procedures, including the at least one result or action taken by the controller and at least one corresponding outcome.

In Example 11, the subject matter of Examples 1-10 optionally includes, wherein the surgical procedure comprises one of a minimally invasive sports medicine procedure or a joint replacement procedure.

In Example 12, the subject matter of Examples 1-11 optionally includes, wherein the one or more cameras are configured for sensing at an infrared wavelength range, wherein using the data sensed by the one or more cameras, the controller is configured to identify at least one of: a change in temperature of the surgical site or the presence of bleeding.

Example 13 is a method of controlling a cuff configured to perform occlusion of a limb of a patient during a surgical procedure, optionally comprising: visualizing a surgical site at the limb of the patient; determining, with a computer, a presence or an absence of bleeding at the surgical site based upon the visualizing; and controlling the cuff to increase or decrease of a pressure of the cuff based upon the determining.

In Example 14, the subject matter of Example 13 optionally includes, sensing an arterial blood pressure at the limb of the patient; and controlling the cuff to increase or decrease the pressure of the cuff in response to data from the sensing in addition to the visualizing.

In Example 15, the subject matter of Examples 13-14 optionally includes, wherein, if the presence of bleeding is determined, increasing the pressure of the cuff by between 10% and 25%, inclusive, and redetermining based upon second data from visualizing the surgical site gathered after an elapsed period of time, the presence or the absence of bleeding at the surgical site.

In Example 16, the subject matter of Examples 13-15 optionally includes, wherein, if the absence of bleeding is determined, decreasing the pressure of the cuff by between 10% and 25%, inclusive, and redetermining based upon second data from visualizing the surgical site gathered after an elapsed period of time, the presence or the absence of bleeding at the surgical site.

In Example 17, the subject matter of Examples 13-16 optionally includes, wherein determining the presence or absence of bleeding is by determining a percent of red color pixels at the surgical site and a rate of change of the red color pixels.

In Example 18, the subject matter of Examples 13-17 optionally includes, selectively controlling fluid communication of an irrigating fluid to or from the surgical site based upon the determined presence or absence of bleeding at the surgical site.

In Example 19, the subject matter of Example 18 includes, wherein the controlling the cuff to increase or decrease of the pressure of the cuff and the selectively controlling fluid communication of the irrigating fluid to or from the surgical site is based at least in part upon a machine learning model.

In Example 20, the subject matter of Examples 13-19 optionally includes, wherein the controlling the cuff to increase or decrease of the pressure of the cuff is based at least in part upon a machine learning model.

In Example 21, the subject matter of Examples 13-20 optionally includes, wherein the surgical procedure comprises one of a minimally invasive sports medicine procedure or a joint replacement procedure.

In Example 22, the subject matter of Examples 13-21 optionally includes, identifying with the computer at least one of: a change in temperature in surgical site or the presence of bleeding using the visualizing.

Example 23 is a surgical system optionally comprising: a tourniquet system including a cuff configured to perform occlusion of a limb of a patient during a surgical procedure; a flow inducing device configured to provide an irrigating fluid to a surgical site at the limb of the patient; and a negative pressure source configured to induce flow to remove the irrigating fluid from the surgical site; a visualization system including one or more cameras configured to provide a field of view that includes, the surgical site; and a controller electronically coupled to the visualization system and the tourniquet system, wherein the controller is configured to determine a presence or an absence of bleeding at the surgical site based upon data from the visualization system, and control at least one of: the tourniquet system to increase or decrease of a pressure of the cuff and fluid communication of the irrigating fluid to or from the surgical site in response to such determination.

In Example 24, the subject matter of Example 23 optionally includes, wherein the controller is configured to: collect and record the data from the visualization system during at least a portion of the surgical procedure; query a database to retrieve information about related prior surgical procedures, the information including at least one result or next action taken after the data from the visualization system was provided to the controller; determine, based upon the information, a next action including at least one of: implementing a change of the pressure of the cuff of the tourniquet system and implementing an addition or removal of the irrigating fluid from the surgical site; implement the next action; and collect further data from at least the visualization system as to results from the next action.

In Example 25, the subject matter of Example 24 optionally includes, a machine learning engine and wherein the controller is configured to train the machine learning engine using the related prior surgical procedures, including the at least one result or action taken by the controller and at least one corresponding outcome.

In Example 26, the subject matter of Examples 23-25 optionally includes, wherein the one or more cameras are configured for sensing at an infrared wavelength range, wherein using the data sensed by the one or more cameras, the controller is configured to identify at least one of: a change in temperature of the surgical site or the presence of bleeding.

In Example 27, the subject matter of Examples 23-26 optionally includes, wherein the tourniquet system includes at least one sensor configured to sense an arterial blood pressure at the limb of the patient, and wherein the controller is configured to control the tourniquet system to increase or decrease the pressure of the cuff in response to data from the at least one sensor in addition to the data from the visualization system.

In Example 28, the subject matter of Examples 23-27 optionally includes, wherein, if the presence of bleeding is determined, the controller is configured to increase the pressure of the cuff by between 10% and 25%, inclusive, and the controller is configured to redetermine based upon second data from the visualization system gathered after an elapsed period of time, the presence or the absence of bleeding at the surgical site.

In Example 29, the subject matter of Examples 23-28 optionally includes, wherein, if the absence of bleeding is determined, the controller is configured to decrease the pressure of the cuff by between 10% and 25%, inclusive, and the controller is configured to redetermine based upon second data from the visualization system gathered after an elapsed period of time, the presence or the absence of bleeding at the surgical site.

In Example 30, the subject matter of Examples 23-29 optionally includes, wherein the controller determines the presence or absence of bleeding based as a percent of red color pixels at the surgical site and a rate of change of the red color pixels.

Example 31 is at least one machine-readable medium including instructions that, when executed by processing circuitry, cause the processing circuitry to perform operations to implement of any of Examples 1-30.

Example 32 is an apparatus comprising means to implement of any of Examples 1-30.

Example 33 is a system to implement of any of Examples 1-30.

Example 34 is a method to implement of any of Examples 1-30.

In Example 35, the apparatuses, methods or systems of any one or any combination of Examples 1-34 can optionally be configured such that all elements or options recited are available to use or select from.

### BRIEF DESCRIPTION OF THE DRAWINGS

Various embodiments of the present invention will now be discussed with reference to the appended drawings. It should be appreciated that the drawings depict only typical embodiments of the invention and are therefore not to be considered limiting in scope.

FIG. 1 is a schematic view of a surgical system that includes a visualization system and a tourniquet system according to an example of the present disclosure.

FIG. 2 a highly schematic view of a surgical system that includes a controller controlling various apparatuses and operations including a cuff according to an example of the present disclosure.

FIG. 3 is a highly schematic view of another surgical system that includes a controller controlling various apparatuses and operations including a cuff according to an example of the present disclosure.

FIG. 4 illustrates a data flow diagram for providing feedback to control a tourniquet system using a visualization system during a total joint arthroplasty in accordance with some embodiments according to an example of the present disclosure.

FIG. 5 illustrates a machine learning engine for determining feedback such as to a tourniquet system according to an example of the present disclosure.

FIG. 6 illustrates a surgical system for intra-operative surgical procedure feedback according to an example of the present disclosure.

FIG. 7 illustrates a flowchart illustrating a technique for providing intra-operative surgical procedure feedback according to an example of the present disclosure.

FIG. 8 illustrates a flowchart illustrating a technique for providing intra-operative surgical procedure feedback in accordance with some embodiments.

FIG. 9 illustrates a block diagram of an example machine upon which any one or more of the techniques, apparatuses, systems or methods discussed herein may perform in accordance with at least one example of this disclosure.

### DETAILED DESCRIPTION OF THE INVENTION

The present invention relates to surgical system that includes a tourniquet system that is responsive to feedback regarding bleeding at the surgical site from a visualization system. Several examples of the surgical system will now be described to provide an overall understanding of the principles of the form, function and methods of use. According to some examples, the systems and methods disclosed can determine and implement an appropriate cuff pressure based upon the presence or absence a surgical procedure based on data collected and analyzed previous surgical procedures are described herein. Recorded of data can then be correlated with the specific patient's outcome including confirmation of presence or absence of blood at the surgical site, time correlations to the presence or absence of blood at the surgical site, applicable sensed cuff pressure(s) during the procedure including during times correlated to the presence and/or absence of blood at the surgical site, sensed arterial blood pressure, sensed systolic pressure, surgical procedure type, patient specific data (anatomical, demographic, etc.), presence of irrigating fluid, amount of irrigating fluid and/or additional information or criteria. Recording and correlation of pre-operative, intra-operative, and post-operative information may then be utilized in real-time to provide evidence based control of the tourniquet system and/or other devices used for procedures encountering similar surgical situations. Such control of the surgical system can be made by a model trained in a machine learning system, or the like. Information collected during a surgical procedure may be automatically stored This description of the general principles of this invention is not meant to limit the inventive concepts in the appended claims.

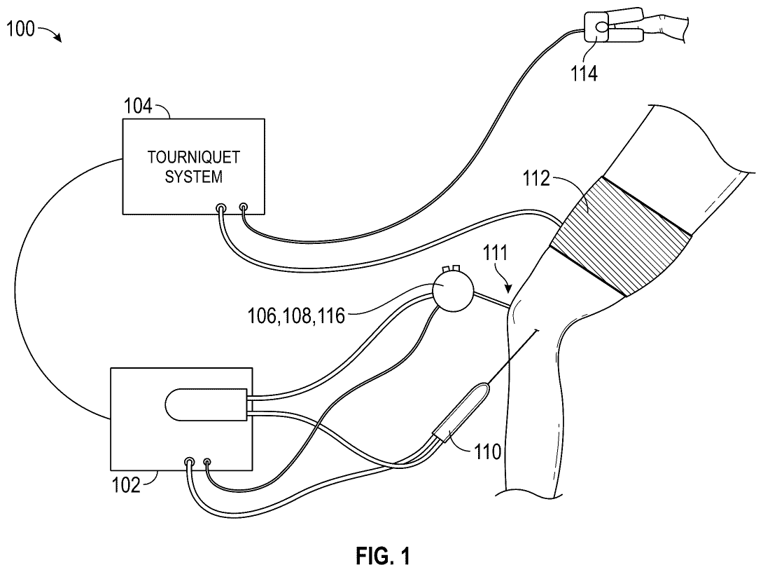

FIG. 1 shows a surgical system 100 including a controller 102, a tourniquet system 104, a visualization system 106, a first surgical device 108 and a second surgical device 110. The tourniquet system 104 can include a cuff 112 and one or more sensors 114.

The controller 102 can be electronically coupled to various of the components and systems including the tourniquet system 104, the visualization system 106, the first surgical device 108 and/or the second surgical device 110 and can control aspects of the operation thereof. The tourniquet system 104 can be coupled to the cuff 112 and the one or more sensors 114.

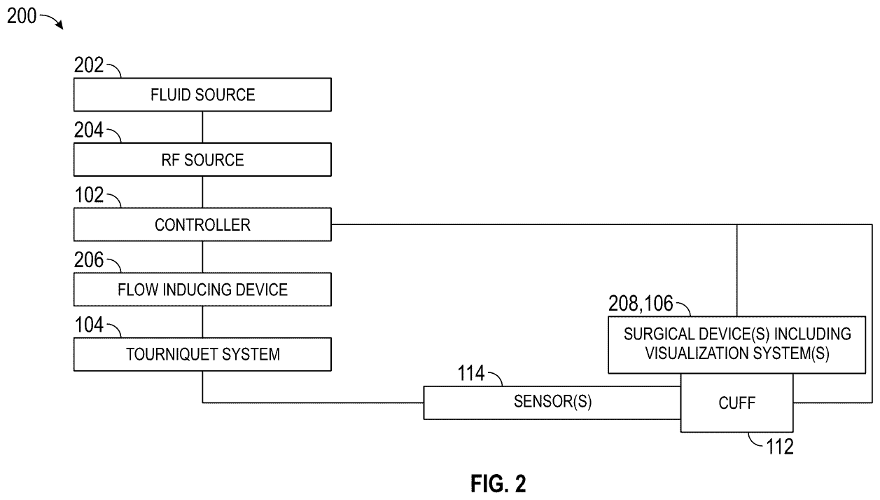

The visualization system 106 of the example of FIG. 2 can be partially integrated into the controller 102 and includes one or more cameras 116 that are carried by the first surgical device 108. However, other examples of the present application contemplate the visualization system 106 can include components separate from the controller 102 (e.g., can utilize a dedicated controller, display(s) or the like). The first surgical device 108 can be an endoscopic device, an arthroscopic cannula as described in Provisional Patent Application 63/588,382 incorporated by reference above, or another suitable device including the one or more cameras 116. The first surgical device 108 and the second surgical device 110 can be inserted into a surgical site 111 adjacent but distal of the cuff 112.

The second surgical device 110 can be an electrosurgical arthroscopic apparatus or other therapeutic device described in the various applications incorporated by reference with the U.S. Application Publications noted above. The second surgical device 110 can be a probe that has a distal (working) end that carries tissue cutting mechanism(s) optionally driven by a drive motor, and RF electrode(s) coupled to an RF source. The RF electrode(s) of the probe can be configured for use in many arthroscopic surgical applications, including but not limited to treating bone in shoulders, knees, hips, wrists, ankles and the spine. Visualization system 106 can in some examples include aspects carried by the second surgical device 110 as described in U.S. Provisional Patent Application 63/588,386 and 63/588,377, incorporated above by reference.

According to one example, the controller 102 can include, for example, software, hardware, and combinations of hardware and software configured to execute several functions related to, among others, operation of the system 100. The controller 102 can be an analog, digital, or combination analog and digital controller including a number of components. As examples, the controller 102 can include integrated circuit boards or ICB(s), printed circuit boards PCB(s), processor(s), data storage devices, switches, relays, or any other components. Examples of processors can include any one or more of a microprocessor, a controller, a digital signal processor (DSP), an application specific integrated circuit (ASIC), a field-programmable gate array (FPGA), or equivalent discrete or integrated logic circuitry. Commercially available microprocessors can be configured to perform the functions of the controller 102. Various known circuits may be associated with controller 102, including power supply circuitry, signal-conditioning circuitry, actuator driver circuitry (i.e., circuitry powering solenoids, motors, or piezo actuators), and communication circuitry. In some examples, the controller 102 may be part of a control unit.

The controller 102 can include a memory such as memory circuitry. The memory may include storage media to store and/or retrieve data or other information such as, operational algorithms. Storage devices, in some examples can be a computer-readable storage medium. The data storage devices can be used to store program instructions for execution by processor(s) of the controller 102, for example. The storage devices, for example, are used by software, applications, algorithms, as examples, running on and/or executed by the controller 102. The storage devices can include short-term and/or long-term memory and can be volatile and/or non-volatile. Examples of non-volatile storage elements include magnetic hard discs, optical discs, floppy discs, flash memories, or forms of electrically programmable memories (EPROM) or electrically erasable and programmable (EEPROM) memories. Examples of volatile memories include random access memories (RAM), dynamic random-access memories (DRAM), static random-access memories (SRAM), and other forms of volatile memories known in the art.

The system 100 can include a flow inducing device (not shown) such as a pump, positive pressure source or the like that is in fluid communication from a fluid source with the second surgical device 110. The flow inducing device can allow for fluid flow to the anatomy being operated upon of an irrigating fluid (e.g., saline) utilized during operation of the second surgical device 110. The system can include a negative pressure source configured to induce flow to remove the irrigating fluid from the surgical site such as via the second surgical device 110. As can be understood from the above description of the system 100, the second surgical device 110, the controller 102 and controller algorithms can be configured to perform and automate many tasks to provide for system functionality including various operation functions of the second surgical device 110. For example, the controller 102 can be configured to selectively control the flow inducing device and the negative pressure source to increase or decrease irrigating fluid at the surgical site based upon the determined presence or absence of bleeding at the surgical site.

By way of example, the controller 102 can configured to control various aspects of the surgery and can be configured to operate the Tricera® System from Zimmer Biomet Inc. The Tricera® System is an advanced arthroscopy system that combines ablation/hemostasis, tissue resection, bone cutting and fluid management into one system.

The tourniquet system 104 can be configured to communicate with the controller 102. The tourniquet system 104 can be, for example, the Zimmer Biomet A.T.S.® 5000, the Zimmer Biomet A.T.S.® 3200, the Zimmer Biomet A.T.S.® 4000, the Zimmer Biomet A.T.S.® 2200, The VasoPress® System or other suitable systems manufactured by applicant. As such, the tourniquet system 104 can have the one or more sensors 114 be configured as Limb Occlusion Pressure (LOP) sensor used by Zimmer Biomet A.T.S.®. The tourniquet system 104 can be designed to calculate a patient's pressure at an individual level and apply the minimum amount of tourniquet pressure via the cuff 112 needed to occlude a limb at a specific time for a specific patient. Thus, the tourniquet system 104 can include at least one sensor (e.g. the one or more sensors 114) configured to sense an arterial blood pressure at the limb of the patient. The controller 102 can be configured to control the tourniquet system 104 to increase or decrease the pressure of the cuff 112 in response to data from the one or more sensors 114 in addition to the data from the visualization system 106.

The visualization system 106 with the one or more cameras 116 can configured to provide a field of view that includes a surgical site at the limb of the patient as shown in FIG. 1. The one or more cameras 116 can be an on-chip controlled devices with the chip being at or adjacent a distal (working) tip of the second surgical device 110. However, other locations for the chip and/or the one or more cameras 116 including off-device locations are contemplated. For example, the one or more cameras 116 can utilize Complementary Metal-Oxide Semiconductor Active Pixel Sensors (CMOS-APS). The CMOS-APS are configured for sensing at an infrared wavelength range, a visual light wavelength range or another wavelength range as desired.

As discussed in further detail herein, the controller 102 is configured to determine a presence or an absence of bleeding at the surgical site based upon data from the visualization system 106, and control the tourniquet system 104 to increase or decrease of a pressure of the cuff 112 in response to such determination.

The visualization system 106 can include at least one of the one or more cameras 116 configured for sensing at an infrared wavelength range. This infrared camera(s) can work in concert with the controller 102, and can be configured to monitor the surgical space (the surgical site 111) for temperature changes caused by radiofrequency devices. An elevated temperature(s) sensed using the infrared camera(s) can be electronically communicated to the controller 102. Based upon such sensed elevated temperature(s), the controller 102 can increase fluid flow of irrigating fluid to the joint, for example. Additionally, or alternatively, the infrared camera(s) can also be used to detect blood such as excessive bleeding by using an algorithm(s) of the controller 102. Such algorithm(s) run on the controller 102 can, for example, be used to identify a temperature difference between the arthroscopic fluid (e.g., saline) and the blood (e.g., about 37 degrees) that can seep from the joint. This difference can be sensed using the infrared camera(s) and identified with suitable algorithm(s). Alternatively, the one or more cameras 116 need not be infrared cameras and the algorithm(s) of the controller 102 can determine the presence or absence of bleeding is by determining a percent of red color pixels at the surgical site, a rate of change of the red color pixels at the surgical site or determining the percent of red color pixels at the surgical site and the rate of change of the red color pixels at the surgical site.

Purely by way of example, if the presence of bleeding is determined, the controller 102 can be configured to increase the pressure of the cuff 112 by between 10% and 25%, inclusive. Additionally, the controller 102 can be configured to redetermine based upon second data from the visualization system 106 gathered after an elapsed period of time, the presence or the absence of bleeding at the surgical site. If the absence of bleeding is determined, the controller 102 can be configured to decrease the pressure of the cuff 112 by between 10% and 25%, inclusive. The controller can be configured to redetermine based upon second data from the visualization system 106 gathered after an elapsed period of time, the presence or the absence of bleeding at the surgical site. However, real time redetermination is also contemplated. The ranges of change to cuff pressure are merely exemplary.

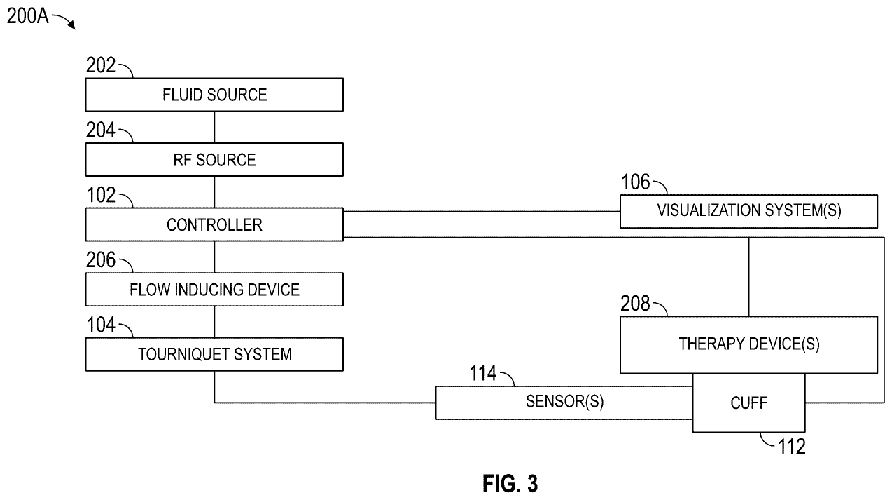

FIGS. 2 and 3 schematically illustrate further surgical systems similar to those of FIG. 1. FIG. 2 shows a surgical system 200 including the controller 102, a fluid source 202 (e.g., for irrigating fluid such as saline), RF source 204, and a flow inducing device 206 (e.g., a pump, a negative pressure source for removing irrigating fluid and other issue removed from the anatomy by operation of the therapeutic device(s)) in addition to the tourniquet system 104 and the visualization system 106. In FIG. 2, it can be seen that the controller 102 is operatively electronically coupled to at least one surgical device 208, the fluid source 202, RF source 204, the flow inducing device 206, the tourniquet system 104 and the visualization system 106. Additionally, the controller 102 can be operatively electronically coupled to control a drive motor (not shown), control communication with the fluid source 202, control communication with the RF source 204, control communication with the flow inducing device and can control the various auxiliary systems or devices (e.g., the one or more sensors 114 and the cuff 112 via the tourniquet system 104, the visualization system 106, etc.). The system 200 can include a display for displaying operating parameters, images that are captured by the visualization system 106, etc.

As shown in FIG. 2, the visualization system 106 can be at least partially integrated into the at least one surgical device 208. Thus, the visualization system 106 can be coupled to and integrated in an arthroscopic device configured for use during the surgical procedure. According to one example, the visualization system 106 can include one or more light sources in addition to one or more cameras 116 (FIG. 1). The one or more light sources can be light emitting diodes (LEDs) or other illumination components. The LED can be an on-chip controlled devices with the chip being at or adjacent a distal (working) tip of the at least one surgical device 208. However, other locations for the chip, the one or more cameras and/or the one or more light sources such as on an elongate shaft proximal of the distal tip are contemplated.

FIG. 3 shows a surgical system 200A having a similar construction to that of the surgical system 200 of FIG. 2. The surgical system 200A thus includes the controller 102, the fluid source 202, the RF source 204, the flow inducing device 206, the tourniquet system 104 and the visualization system 106 as previously discussed. However, the visualization system 106 in the example of FIG. 3 is separate from and not integrated into the at least one surgical device 208 that applies therapy at the surgical site. Thus, the visualization system 106 can include standalone dedicated components (e.g., the one or more cameras, the one or more light sources) that are not part of the at least one surgical device 208 applying therapy. Thus, FIG. 3 is akin to the arrangement of FIG. 1, for example.

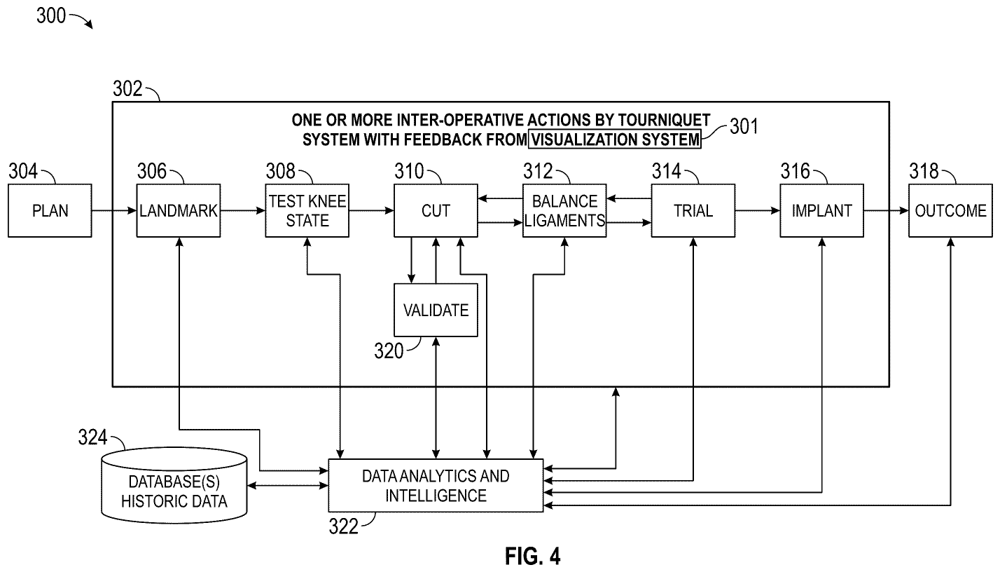

FIG. 4 shows an example of a analytics system 300 used for a total joint or partial joint arthroplasty. The analytics system 300 can include feedback from a visualization system 301 that is used to control a tourniquet system 302 during the total joint or partial joint procedure. During the procedure, such as in the operating room, a data analytic program may be run (e.g., on the controller 102 discussed previously in regards to FIGS. 1-3 or another device). The program may run a simulation in real time using data from the visualization system 301 to autonomously control the tourniquet system 302. The data analytic program insight may be based on statistical analysis of historical data and intra-operative decisions or actions taken by the surgeon, data from the visualization system 301 and/or other criteria such as but not limited to: confirmation of presence or absence of blood at the surgical site, time correlations to the presence or absence of blood at the surgical site, applicable sensed cuff pressure(s) during the procedure including during times correlated to the presence and/or absence of blood at the surgical site, sensed arterial blood pressure, sensed systolic pressure, surgical procedure type, patient specific data (anatomical, demographic, etc.), presence of irrigating fluid, amount of irrigating fluid and/or additional information or criteria. In an example, at any step of the procedure, an intervention using the tourniquet system 302 can be made. Additionally, an alert to the surgeon may be updated based on latest actions. These interventions may result in better patient outcome. The data generated or stored during the procedure may be stored (e.g., on the database) and used in future procedures.

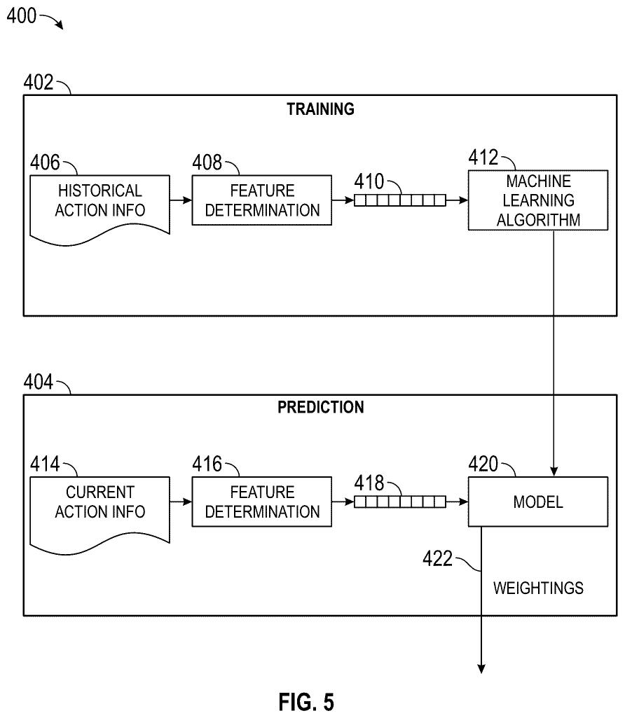

FIG. 5 is a flow diagram for providing feedback to the tourniquet system 302 via the visualization system 301 in accordance with some embodiments. In an example, a plan is created or selected at 304, which may be implemented in whole or in part by one or more surgical devices. The one or more surgical devices may perform or record intra-operative actions, including landmarking 306, testing a state of anatomy (e.g., a knee, ankle, wrist, elbow, etc.) 308, performing a cut 310 (which may be validated at 320), balancing soft tissue (e.g., a ligament of the knee) 312, inserting a trial 314, or inserting an implant 316, which may result in an outcome 318. These operations 306-320 along with data from the visualization system 301 and/or data from the tourniquet system 302 may be sent to a data analytics and intelligence engine 322 for determining one or more correlations, one or more causations, or other analytical information from actions taken by or recorded by the visualization system 301 and the tourniquet system 302 compared to outcomes 318. In an example, the data analytics and intelligence engine 322 may store information in a database 324 or retrieve historical data from the database 324, such as when determining a presence or an absence of bleeding at the surgical site based upon data from the visualization system 301. This can be used to control the tourniquet system 302 to increase or decrease of a pressure of a cuff in response to such determination and/or the other analytical information. In an example, the operations 306-320 may be sent to directly to the database 324, stored in memory of the controller, or stored on a portable memory device or other medium.

After some amount of historical data is stored (e.g., at the database 324), the data analytics and intelligence engine 322 may work autonomously or semi-autonomously to control the tourniquet system 302 based upon data from the visualization system 301 but also additionally based upon historical data. For example, the historical data (stored in database 324) may be used to compare actions taken (e.g., intra-operative actions) to outcomes 318. The comparison may be performed by a user or via machine learning techniques. The analytics system 300 may provide real-time feedback to a surgeon. The system 300 (e.g., the controller or other suitably configured electronic device) may evaluate information being collected in the current procedure against historical data correlated to positive outcomes. For example, at operations such as landmarking 306 the surgeon uses the visualization system 301 or other device(s) to collect a set of landmarks from the actual anatomy that allows for correlation of the pre-operative plan with the specific patient anatomy. While the pre-operative plan was developed from medical images of the specific patient's anatomy, medical imaging is not perfect. Accordingly, the system 300 may determine how well the landmarks collected intra-operatively align with the pre-operative plan. Additionally, machine learning algorithms may evaluate not only how the collected landmarks align with the pre-operative plan in comparison to historically good procedures, but also evaluate how the landmarks themselves compare to previous procedures with bleeding outcomes. For example, the system may determine that certain landmarks exhibit a particular spatial relationship that historically has required a certain modification to cuff pressure and/or saline volume to obtain a positive patient outcome (e.g., reduced or absence or bleeding). In such situations, the system 300 can autonomously or semi-autonomously make a modification to the cuff pressure and/or saline volume based on the correlation.

The data analytics and intelligence engine 322 may develop an intervention (e.g., adjustment to cuff pressure, adjustment to saline volume or combination thereof based on historical data saved in the database 324, after tying the historical data to the outcome 318. In an example, intervention may be determined by extrapolating or interpolating information from the historical data or the outcome 318. For example, the data analytics and intelligence engine 322 may run a simulation or apply a machine learning technique to generate additional data, such as by using the historical data or the outcome 318 as training data for the simulation or the machine learning technique. For example, in the abnormal landmark location example, the system may utilize a linear regression technique to extrapolate an appropriate adjustment to the cuff pressure based on historical data from procedures with a positive outcome.

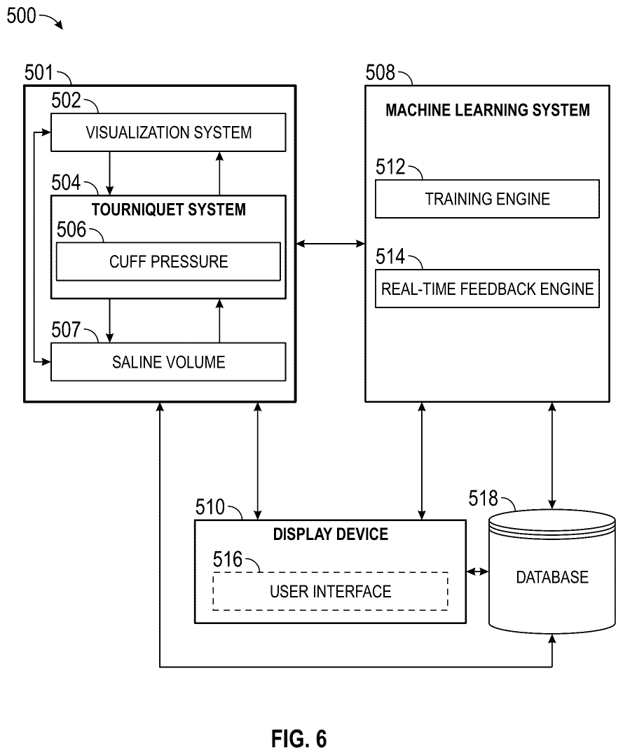

FIG. 5 illustrates a machine learning engine for determining feedback to tourniquet system in accordance with some embodiments. A system may calculate one or more weightings for criteria based upon one or more machine learning algorithms. FIG. 4 shows an example machine learning engine 400 according to some examples of the present disclosure. Machine learning engine 400 may be part of the system 100 of FIG. 1 or system 300 of FIG. 4, for example implemented using a database, a server, etc., or the machine learning system 508 of FIG. 6, described below.

Machine learning engine 400 utilizes a training engine 402 and a prediction engine 404. Training engine 402 inputs historical transaction information 406 for historical actions of stored or generated at a robotic surgical device, for example, into feature determination engine 408. The historical action information 406 may be labeled with an indication, such as a degree of success of an outcome of a surgical procedure, which may include pain information, patient feedback, implant success, ambulatory information, bleeding or lack of bleeding, or the like. In some examples, an outcome may be subjectively assigned to historical data, but in other examples, one or more labelling criteria may be utilized that may focus on objective outcome metrics (e.g., presence or absence of bleeding, pain rating, survey score, a patient satisfaction score, such as a forgotten knee score, a WOMAC score, shoulder assessment, hip assessment, or the like).

Feature determination engine 408 determines one or more features 410 from this historical information 406. Stated generally, features 410 are a set of the information input and is information determined to be predictive of a particular outcome. Example features are given above including cuff pressure setting(s), saline volume use, timing of changes to cuff pressure and/or saline volume, other sensor derived input(s), etc. In some examples, the features 410 may be all the historical activity data, but in other examples, the features 410 may be a subset of the historical activity data. The machine learning algorithm 412 produces a model 420 based upon the features 410 and the labels.

In the prediction engine 404, current action information 414 (e.g., sensor inputs including visualization data or the like) may be input to the feature determination engine 416. Feature determination engine 416 may determine the same set of features or a different set of features from the current information 414 as feature determination engine 408 determined from historical information 406. In some examples, feature determination engine 416 and 408 are the same engine. Feature determination engine 416 produces feature vector 418, which is input into the model 420 to generate one or more criteria weightings 422. The training engine 402 may operate in an offline manner to train the model 420. The prediction engine 404, however, may be designed to operate in an online manner. It should be noted that the model 420 may be periodically updated via additional training or user feedback (e.g., an update to a technique or procedure).

The machine learning algorithm 412 may be selected from among many different potential supervised or unsupervised machine learning algorithms. Examples of supervised learning algorithms include artificial neural networks, Bayesian networks, instance-based learning, support vector machines, decision trees (e.g., Iterative Dichotomiser 3, C4.5, Classification and Regression Tree (CART), Chi-squared Automatic Interaction Detector (CHAD), and the like), random forests, linear classifiers, quadratic classifiers, k-nearest neighbor, linear regression, logistic regression, and hidden Markov models. Examples of unsupervised learning algorithms include expectation-maximization algorithms, vector quantization, and information bottleneck method. Unsupervised models may not have a training engine 402. In an example embodiment, a regression model is used and the model 420 is a vector of coefficients corresponding to a learned importance for each of the features in the vector of features 410, 418.

FIG. 6 illustrates a system 500 for intra-operative surgical procedure feedback based upon a visualization system 502 including for control of tourniquet system 504 in accordance with some embodiments. The system 500 includes a surgical system 501, which may include the visualization system 502, the tourniquet system 504 with input control over a cuff pressure 506, a surgical tool (not shown), a saline volume 507, or the like. The visualization system 502 and other components of the system 500 such as the tourniquet system 504, surgical device(s), saline volume 507 may output data to a machine learning system 508, a display device 510, and/or a database 518. In an example, the machine learning system 508 may output information to the display device 510 and the database 518. The display device may retrieve information stored in the database 518. The display device 510 may be used to display a user interface 516. In an example, the machine learning system 508 includes a training engine 512 and a real-time feedback engine 514.

The surgical system 501 may be used to perform all or a portion of a surgical procedure on a patient. A processor may be coupled to memory (e.g., on the surgical system 501 or the machine learning system 508). The processor may be used to record an action taken by surgical device (not shown), the tourniquet system 504, with data from the visualization system 502 such as during the portion of the surgical procedure. The processor may query a database to retrieve information about related prior surgical procedures. In an example, the information may include at least one result or next action taken after the action (e.g., adjust cuff pressure 506, adjust saline volume 507, adjust both). The processor may determine a recommended change, such as based on the information, to alter the cuff pressure, alter the saline volume or change another aspect of the surgical procedure. The recommended change may be a change as performed by the tourniquet system 504 or another component of the surgical system 501 as previously reviewed in the prior examples of FIGS. 1-5. The processor may control the change and can also output the change (e.g., to the display device 510). The change may include using the processor to intraoperatively implement alteration of the cuff pressure 506, saline volume 507, or both. Thus, the output (the change) may be performed without surgeon input as an alert.

The machine learning system 508 may train using the related prior surgical procedures, including, for example, at least one action taken by the tourniquet system 504 or at least one corresponding outcome. The at least one corresponding outcome may be based on a patient outcome received from the patient. In an example, the processor may submit a plan to the machine learning system 508 to receive feedback preoperatively or intraoperatively. In an example, the machine learning system 508 may simulate at least a portion of the surgical procedure to determine changes. The machine learning system 508 may select the change/changes from a plurality of possible changes, such as based on outcome likelihoods of the plurality of possible changes. the information about related prior surgical procedures may include patient-specific information about a past procedure performed on the patient (e.g., during a revision surgery, Information can include various sensor information discussed above, surgical procedure type information, demographic-specific information corresponding to the patient, etc. The demographic-specific information may include at least one of patient size (e.g., height, weight, gender, which knee, hip, or shoulder, etc.), patient age, or the like.

The system 500 such as via a controller (e.g., the controller 102 discussed previously) can collect and record the data from the visualization system 502 during at least a portion of the surgical procedure. The system 500 can query a database to retrieve information about related prior surgical procedures, the information including at least one result or next action taken after the data from the visualization system 502 was provided to the controller. The system 500 can determine, based upon the information, a next action including at least one of: implementing a change of the pressure of the cuff of the tourniquet system 504 and implementing an addition or removal of the irrigating fluid from the surgical site. The system 500 can implement the next action and collect further data from at least the visualization system 502 as to results from the next action.

Alternatively, the system 500 may only implement some changes and not others. Thus, for example, the system 500 can collect and record the data from the visualization system 502 during at least portion of the surgical procedure. The system 500 can query a database to retrieve information about related prior surgical procedures, the information including at least one result or next action taken after the data from the visualization system 502 was provided to the controller. The system 500 can determine, based upon the information, a change of the pressure of the cuff of the tourniquet system 504. The system 500 can implement the change and collect further data from at least the visualization system 502 and redetermine the presence or absence of bleeding as a result of the change.

Further alternatives contemplated would be that the system 500 can determine, based upon the information, a next action including only implementing an addition or removal of the irrigating fluid from the surgical site while no change is made to the pressure of the cuff.

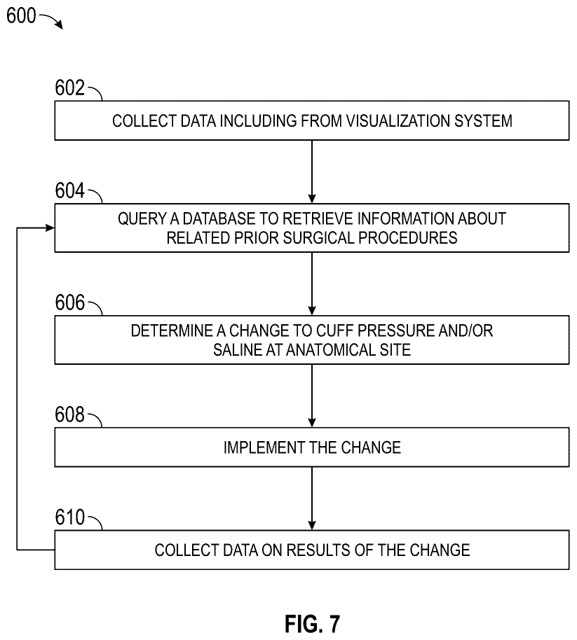

FIG. 7 illustrates a flow chart of a method 600 of controlling a cuff configured to perform occlusion of a limb of a patient during a surgical procedure such as by using aspects of the system 500 discussed previously in regard to FIG. 6. The method 600 can include collecting 602 information (data) including data from the visualization system. The method 600 can include querying 604 a database to retrieve information about related prior procedures. The method 600 can include determining 606 a change to a cuff pressure and/or saline volume at a surgical site. The method 600 can implement 608 this change. The method 600 can collect 610 data (e.g., visualization data, other sensor data, etc.) on results of the change.

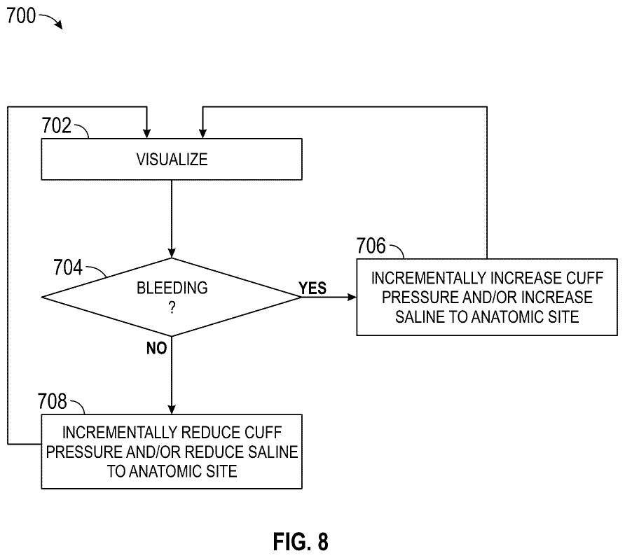

FIG. 8 shows a flow diagram of another method 700 of controlling a cuff configured to perform occlusion of a limb of a patient during a surgical procedure. The method 700 need not use data analytics or machine learning techniques discussed previously including in reference to the method of FIG. 7. The method 700 can include visualizing a surgical site at the limb of the patient at 702. The method 700 can include determining 704, with a computer, a presence or an absence of bleeding at the surgical site based upon the visualizing. The method can include controlling the cuff to increase 706 or decrease 708 of a pressure of the cuff based upon the determining.

Optionally the method 700 can include sensing an arterial blood pressure at the limb of the patient and controlling the cuff to increase or decrease the pressure of the cuff in response to data from the sensing in addition to the visualizing. Optionally, if the presence of bleeding is determined, the method can include increasing the pressure of the cuff by between 10% and 25%, inclusive, and redetermining based upon second data from visualizing the surgical site gathered after an elapsed period of time, the presence or the absence of bleeding at the surgical site. Optionally, if the absence of bleeding is determined, the method can include decreasing the pressure of the cuff by between 10% and 25%, inclusive, and redetermining based upon second data from visualizing the surgical site gathered after an elapsed period of time, the presence or the absence of bleeding at the surgical site. As an example, the determining the presence or absence of bleeding is by determining a percent of red color pixels at the surgical site, a rate of change of the red color pixels at the surgical site or determining the percent of red color pixels at the surgical site and the rate of change of the red color pixels at the surgical site. Optionally, the method 700 can include selectively controlling fluid communication of an irrigating fluid to or from the surgical site based upon the determined presence or absence of bleeding at the surgical site. The controlling the cuff to increase or decrease of the pressure of the cuff and the selectively controlling fluid communication of the irrigating fluid to or from the surgical site can be based at least in part upon a machine learning model such as those previously discussed. According to another example, at least the controlling the cuff to increase or decrease of the pressure of the cuff can be based at least in part upon a machine learning model such as those previously discussed. As discussed an illustrated herein, the surgical procedure can comprise one of a minimally invasive sports medicine procedure or a joint replacement procedure. The method 700 can include identifying with the computer at least one of: a change in temperature in surgical site or the presence of bleeding using the visualizing.

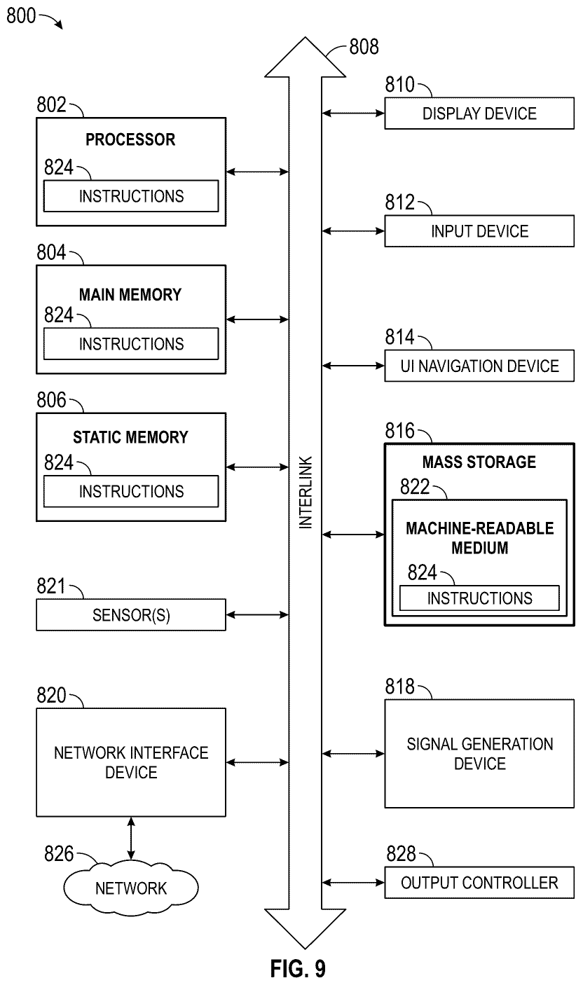

FIG. 9 illustrates a block diagram of an example machine 800 upon which any one or more of the techniques discussed herein may perform in accordance with some embodiments. This example machine can operate some or all of the apparatus and/or system function discussed herein. In other examples, the example machine 800 is merely one of many such machines utilized. In alternative embodiments, the machine 800 may operate as a standalone device or may be connected (e.g., networked) to other machines. In a networked deployment, the machine 800 may operate in the capacity of a server machine, a client machine, or both in server-client network environments. In an example, the machine 800 may act as a peer machine in peer-to-peer (P2P) (or other distributed) network environment. The machine 800 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 800 may include a hardware processor 802 (e.g., a central processing unit (CPU), a graphics processing unit (GPU), a hardware processor core, or any combination thereof), a main memory 804 and a static memory 806, some or all of which may communicate with each other via an interlink (e.g., bus) 808. The machine 800 may further include a display unit 810, an alphanumeric input device 812 (e.g., a keyboard), and a user interface (UI) navigation device 814 (e.g., a mouse). In an example, the display unit 810, input device 812 and UI navigation device 814 may be a touch screen display. The machine 800 may additionally include a storage device (e.g., drive unit) 816, a signal generation device 818 (e.g., a speaker), a network interface device 820, and plurality of sensors 821, such as any of those discussed previously (e.g., an IMU, a global positioning system (GPS) sensor, compass, accelerometer, or other sensor). The machine 800 may include an output controller 828, such as a serial (e.g., Universal Serial Bus (USB), parallel, or other wired or wireless (e.g., infrared (IR), near field communication (NFC), etc.) connection to communicate or control one or more peripheral devices (e.g., a printer, card reader, etc.).

The storage device 816 may include a machine readable medium 822 on which is stored one or more sets of data structures or instructions 824 (e.g., software) embodying or utilized by any one or more of the techniques or functions described herein. The instructions 824 may also reside, completely or at least partially, within the main memory 804, within static memory 806, or within the hardware processor 802 during execution thereof by the machine 800. In an example, one or any combination of the hardware processor 802, the main memory 804, the static memory 806, or the storage device 816 may constitute machine readable media.

While the machine readable medium 822 is illustrated as a single medium, the term “machine readable medium” may include a single medium or multiple media (e.g., a centralized or distributed database, and/or associated caches and servers) configured to store the one or more instructions 824. The term “machine readable medium” may include any medium that is capable of storing, encoding, or carrying instructions for execution by the machine 800 and that cause the machine 800 to perform any one or more of the techniques of the present disclosure, or that is capable of storing, encoding or carrying data structures used by or associated with such instructions. Non-limiting machine-readable medium examples may include solid-state memories, and optical and magnetic media.

The instructions 824 may further be transmitted or received over a communications network 826 using a transmission medium via the network interface device 820 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 820 may include one or more physical jacks (e.g., Ethernet, coaxial, or phone jacks) or one or more antennas to connect to the communications network 826. In an example, the network interface device 820 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 800, and includes digital or analog communications signals or other intangible medium to facilitate communication of such software

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The term “substantially”, “generally” or “about” mean within 15% of the value provided. The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Method and system examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

All references, including publications, patent applications, and patents, cited herein are hereby incorporated by reference to the same extent as if each reference were individually and specifically indicated to be incorporated by reference and were set forth in its entirety herein.

What is claimed is:

1. A surgical system comprising:
a tourniquet system including a cuff configured to perform occlusion of a limb of a patient during a surgical procedure;
a visualization system including one or more cameras configured to provide a field of view that includes a surgical site at the limb of the patient; and
a controller electronically coupled to the visualization system and the tourniquet system, wherein the controller is configured to determine a presence or an absence of bleeding at the surgical site based upon data from the visualization system, and control the tourniquet system to increase or decrease of a pressure of the cuff in response to such determination.

2. The surgical system of claim 1, wherein the tourniquet system includes at least one sensor configured to sense an arterial blood pressure at the limb of the patient, and wherein the controller is configured to control the tourniquet system to increase or decrease the pressure of the cuff in response to data from the at least one sensor in addition to the data from the visualization system.

3. The surgical system of claim 1, wherein the visualization system is coupled to and integrated in an arthroscopic device configured for use during the surgical procedure.

4. The surgical system of claim 1, wherein, if the presence of bleeding is determined, the controller is configured to increase the pressure of the cuff by between 10% and 25%, inclusive, and the controller is configured to redetermine based upon second data from the visualization system gathered after an elapsed period of time, the presence or the absence of bleeding at the surgical site.

5. The surgical system of claim 1, wherein, if the absence of bleeding is determined, the controller is configured to decrease the pressure of the cuff by between 10% and 25%, inclusive, and the controller is configured to redetermine based upon second data from the visualization system gathered after an elapsed period of time, the presence or the absence of bleeding at the surgical site.

6. The surgical system of claim 1, wherein the controller determines the presence or absence of bleeding based as a percent of red color pixels at the surgical site and a rate of change of the red color pixels.

7. The surgical system of claim 1, further comprising:
a flow inducing device configured to provide an irrigating fluid to the surgical site; and
a negative pressure source configured to induce flow to remove the irrigating fluid from the surgical site;
wherein the controller is configured to selectively control the flow inducing device and the negative pressure source to increase or decrease irrigating fluid at the surgical site based upon the determined presence or absence of bleeding at the surgical site.

8. The surgical system of claim 7, wherein the controller is configured to:
collect and record the data from the visualization system during at least a portion of the surgical procedure;
query a database to retrieve information about related prior surgical procedures, the information including at least one result or next action taken after the data from the visualization system was provided to the controller;
determine, based upon the information, a next action including at least one of: implementing a change of the pressure of the cuff of the tourniquet system and implementing an addition or removal of the irrigating fluid from the surgical site;
implement the next action; and
collect further data from at least the visualization system as to results from the next action.

9. The surgical system of claim 1, wherein the controller is configured to:
collect and record the data from the visualization system during at least portion of the surgical procedure;
query a database to retrieve information about related prior surgical procedures, the information including at least one result or next action taken after the data from the visualization system was provided to the controller;
determine, based upon the information, a change of the pressure of the cuff of the tourniquet system;
implement the change; and
collect further data from at least the visualization system and redetermine the presence or absence of bleeding as a result of the change.

10. The surgical system of claim 9, further comprising a machine learning engine and wherein the controller is configured to train the machine learning engine using the related prior surgical procedures, including the at least one result or action taken by the controller and at least one corresponding outcome.

11. The surgical system of claim 1, wherein the surgical procedure comprises one of a minimally invasive sports medicine procedure or a joint replacement procedure.

12. The surgical system of claim 1, wherein the one or more cameras are configured for sensing at an infrared wavelength range, wherein using the data sensed by the one or more cameras, the controller is configured to identify at least one of: a change in temperature of the surgical site or the presence of bleeding.

13. A method of controlling a cuff configured to perform occlusion of a limb of a patient during a surgical procedure, comprising:
visualizing a surgical site at the limb of the patient;
determining, with a computer, a presence or an absence of bleeding at the surgical site based upon the visualizing; and
controlling the cuff to increase or decrease of a pressure of the cuff based upon the determining.

14. The method of claim 13, further comprising:
sensing an arterial blood pressure at the limb of the patient; and
controlling the cuff to increase or decrease the pressure of the cuff in response to data from the sensing in addition to the visualizing;
wherein determining the presence or absence of bleeding is by determining a percent of red color pixels at the surgical site and a rate of change of the red color pixels.

15. The method of claim 13, wherein at least one of: if the presence of bleeding is determined, increasing the pressure of the cuff by between 10% and 25%, inclusive, and redetermining based upon second data from visualizing the surgical site gathered after an elapsed period of time, the presence or the absence of bleeding at the surgical site; or, if the absence of bleeding is determined, decreasing the pressure of the cuff by between 10% and 25%, inclusive, and redetermining based upon second data from visualizing the surgical site gathered after an elapsed period of time, the presence or the absence of bleeding at the surgical site.

16. The method of claim 13, further comprising selectively controlling fluid communication of an irrigating fluid to or from the surgical site based upon the determined presence or absence of bleeding at the surgical site, wherein the controlling the cuff to increase or decrease of the pressure of the cuff and the selectively controlling fluid communication of the irrigating fluid to or from the surgical site is based at least in part upon a machine learning model.

17. The method of claim 16, further comprising identifying with the computer at least one of: a change in temperature at the surgical site or the presence of bleeding using the visualizing.

18. A surgical system comprising:
a tourniquet system including a cuff configured to perform occlusion of a limb of a patient during a surgical procedure;
a flow inducing device configured to provide an irrigating fluid to a surgical site at the limb of the patient; and
a negative pressure source configured to induce flow to remove the irrigating fluid from the surgical site;
a visualization system including one or more cameras configured to provide a field of view that includes the surgical site; and
a controller electronically coupled to the visualization system and the tourniquet system, wherein the controller is configured to determine a presence or an absence of bleeding at the surgical site based upon data from the visualization system, and control at least one of: the tourniquet system to increase or decrease of a pressure of the cuff and fluid communication of the irrigating fluid to or from the surgical site in response to such determination.

19. The surgical system of claim 18, wherein the controller is configured to:
collect and record the data from the visualization system during at least a portion of the surgical procedure;
query a database to retrieve information about related prior surgical procedures, the information including at least one result or next action taken after the data from the visualization system was provided to the controller;
determine, based upon the information, a next action including at least one of: implementing a change of the pressure of the cuff of the tourniquet system and implementing an addition or removal of the irrigating fluid from the surgical site;
implement the next action; and
collect further data from at least the visualization system as to results from the next action.

20. The surgical system of claim 19, wherein the one or more cameras are configured for sensing at an infrared wavelength range, wherein using the data sensed by the one or more cameras, the controller is configured to identify at least one of: a change in temperature of the surgical site or the presence of bleeding, wherein the tourniquet system includes at least one sensor configured to sense an arterial blood pressure at the limb of the patient, and wherein the controller is configured to control the tourniquet system to increase or decrease the pressure of the cuff in response to data from the at least one sensor in addition to the data from the visualization system.
